(19) United States
(12) Patent Application Publication
(10) Pub. No.: US 2020/0408775 A1
(43) Pub. Date: Dec. 31, 2020

(54) APPARATUS AND METHOD FOR MULTIPLEXED PROTEIN QUANTIFICATION

(71) Applicant: BIOMOTIF AB, Danderyd (SE)

(72) Inventors: Juan ASTORGA WELLS, Stockholm (SE); Roman ZUBAREV, Stockholm (SE); Thorleif LAVOLD, Stocksund (SE)

(21) Appl. No.: 16/976,726

(22) PCT Filed: Mar. 8, 2019
(86) PCT No.: PCT/EP2019/055858

§ 371 Date: Aug. 28, 2020

(30) Foreign Application Priority Data
Mar. 9, 2018 (SE) 1850256-7

(51) Int. Cl. G01N 33/68; C12N 9/76; C07K 1/28; G01N 27/447

(52) U.S. Cl. CPC G01N 33/6848; C12N 9/6427; G01N 2560/00; G01N 27/44795; C07K 1/28

(57) ABSTRACT

The present disclosure provides a method and apparatus for improvements of sample throughput in proteome analysis by mass spectrometry, by combining multiple non-overlapping isoelectric focusing separations. The method for performing an analysis of a plurality of protein samples, comprises: (a) Adding a proteolytic enzyme of a given specificity to a first protein sample to digest proteins to peptides; (b) Separating the peptides obtained in step (a) by isoelectric focusing; (c) Collecting those peptides which have their isoelectric point value within a first isoelectric point range; (d) Adding a proteolytic enzyme of a given specificity to a second protein sample to digest proteins to peptides; (e) Separating the peptides obtained in step (d) by isoelectric focusing; (f) Collecting those peptides which have their isoelectric point value within a second isoelectric point range, where said second isoelectric point range is different and non-overlapping compared to said first isoelectric point range; (g) Combining the peptides collected in steps (c) and (f) into a single sample and subjecting said sample to mass spectrometry analysis; (h) Deconvoluting signals/data obtained from the mass spectrometry analysis by calculating the isoelectric point of each peptide, and assigning a peptide to the first protein sample if its isoelectric point value matches the isoelectric point range selected in step (c) or to the second protein sample if its isoelectric point value matches the isoelectric point range selected in step (f); and (i) Obtaining quantitative information for proteins of each sample according to magnitude of the signal obtained from each peptide.

### FIELD OF THE DISCLOSURE

The present disclosure is related to the field of proteome analysis by mass spectrometry. This field utilizes a series of analytical protocols that are well-known-in-the-art to identify and quantify proteins. The proteins could come from biofluids, cell or microorganisms cultures, biopsies, single expressed proteins, biosimilars or food sources.

### BACKGROUND OF THE DISCLOSURE

Mass spectrometry (MS) remains the main technique for large scale characterization and quantification of proteins. Decades of advances in MS instrumentation, bioinformatics and separation technology have allowed routine quantification of thousands of proteins from cell cultures or human tissues. Furthermore, extensive fractionation and long separation times allows the analysis of full proteomes (10,000 to 12,000 proteins). These advances are likely to continue as mass spectrometers manufacturers constantly release to the market novel instruments with improved sensitivity, speed and resolution.

In brief, proteome analysis involves protein extraction, solubilization, reduction/alkylation, digestion, separation and mass spectrometry analysis. Many variations of this workflow are well known in the art and available in scientific literature.

Limitations of Mass Spectrometry-Based Proteomics

Although MS analysis provides an unmatched proteome depth (number of identified proteins), its sample throughput remains low. Additionally, the cost of MS instrumentation and maintenance is rather high compared to other techniques. For example antibody-based protein quantification, which measures a single (or few) protein(s) per assay is widely used because it is less expensive, easier to implement, highly parallelizable. Furthermore, antibody-based protein quantification has a simpler sample preparation procedure and an easier read-out signal than MS.

Thus for MS, sample multiplexing has been regarded as an important step for expanding its utilization in routine protein analysis (e.g. clinical diagnosis) by means of increasing sample throughput (lower cost-per-analysis).

Sample Multiplexing in Mass Spectrometry

Sample multiplexing refers to mass spectrometry-related methods using a signal convolution/deconvolution process. These methods utilize a signal convolution/deconvolution process to analyze a plurality of samples in less analytical steps than individually analyzing each particular sample. In other words, multiplexing methods decrease the number of assays required to analyze a given number of samples, by allowing mixing a plurality of samples, thus decreasing the number of analyses required to run all said plurality of samples.

In general terms, sample multiplexing by MS involves a three-steps process: signal convolution, mass spectrometrical analysis and signal deconvolution.

Traditional methods for signal convolution can be achieved by chemically modifying the analytes of interest in a manner that can be later detected by the mass spectrometer. This is normally done by a chemical modification that does not dramatically change the physico-chemical characteristics of the analytes, but provides a measurable mass shift that allows determining its origin, in other words the chemical modification allows to determine the signal origin when combined with other samples.

One manner to increase sample throughput is by isobaric labelling. This method utilizes a repertoire of molecules (tags) that have the same mass when intact, but generate fragments (reporter ions) with different masses when fragmented. These molecules contain four regions: a mass reporter region, a cleavable linker region, a mass normalization region and an amine-reactive group. The chemical structures of all the tags are identical but each contains isotopes substituted at various positions, such that the mass reporter and mass normalization regions have different molecular masses in each tag. In this manner, each tryptic peptide sample is labeled with a different isobaric labelling tag, and then all samples are combined/pooled and analyzed by MS/MS. During the LC MS analysis, each tryptic peptide is fragmented by tandem mass spectrometry (CID or HCD). The fragmentation generates tandem mass spectra (MS2 spectra) where the mass and intensity of the different reporter ions coming from each individual sample can be measured. Since the signal intensity of each reporter ion is related to the peptide concentration in each individual sample, the protein abundance from where the peptide can be calculated by measuring the intensity of the particular reporter ion.

In a sense, the fragmentation “releases” the quantitative information encoded into each peptide, which can later be correlated to the abundance of the protein from which the peptide originated and its respective sample.

The use of isobaric labelling, either iTRAQ or TMT, is well known in the art and it has been widely described in scientific literature.

### SUMMARY OF THE DISCLOSURE

The present disclosure provides a method and apparatus for the further improvements of sample throughput in proteome analysis by mass spectrometry. These improvements are achieved by a method and an apparatus according to the independent claims. Preferred embodiments are set forth in the dependent claims.

### BRIEF DESCRIPTION OF THE DRAWINGS

FIG. 1. Schematic drawing illustrating a pI-multiplexing workflow for 3-samples multiplexing. FIG. 1(a) is the pI-code multiplexed method according to the present disclosure while FIG. 1(b) is a standard analysis method according to prior art, in which no multiplexing is done.

FIG. 2. Histogram of pI distributions of tryptic peptides depicting the presence of acid, neutral and basic channel that can be used for pI-multiplexing.

FIG. 3. pH profile of a Non-linear Immobilized pH Gradient Strip (IPG Strip) for pI-Code Multiplexing. This configuration can be used for separation and collection of acid, neutral and basic channels for pI-multiplexing.

FIG. 4. An isoelectric focusing (IEF) device dedicated to isolate the Acid Channel, the Neutral Channel and the Basic Channel for pI-based multiplexing.

FIG. 5. Volcano plot—log10(p-value) vs. log2(Treated/Control ratio)—depicting changes in protein abundance between control and treated sample by use of 3-channel pI-multiplexing.

FIG. 6. Schematics and operational characteristics of a device dedicated to direct isolation of three pI-multiplexing channels.

### DETAILED DESCRIPTION OF THE DISCLOSURE

Multiplexing—Encode the Sample Origin in a Manner That Can Be Later Deconvoluted

In the present disclosure, signal convolution is achieved by encoding the information of the sample origin in discrete isoelectric point ranges of the digested peptides. In other words, each sample has a particular isoelectric point range, thus when combined the origin of the signal can be obtained by calculating the isoelectric point of the peptides. In this manner, a convoluted sample consisting in a mixture of proteolytic peptides coming from a plurality of samples are analyzed and the signal can be deconvoluted (determine its origin) by obtaining the isoelectric point of each peptide.

In a further improvement, isoelectric-point multiplexing is combined with isobaric labelling and/or enzyme multiplexing. Enzyme multiplexing is achieved by using proteolytic enzymes with non-overlapping specificities (the information of sample origin information is encoded into the N-terminus or C-terminus of the resulting proteolytic peptide).

In this manner, the information of the sample origin can be encoded in the pI value as well as in the N-terminus or C-terminus amino acid residue of the digested peptide.

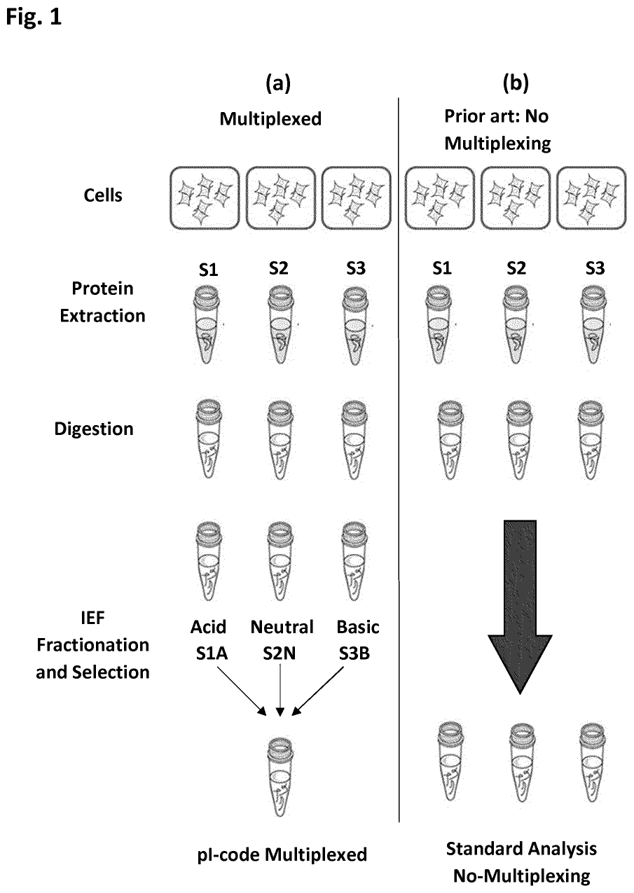

In the present disclosure, signal convolution is achieved by encoding the information of the sample origin into a physicochemical property of the sample (isoelectric point values). Signal deconvolution is achieved by calculating the theoretical pI value based on the polypeptide sequence. The knowledge of the polypeptide sequence is obtained from tandem mass spectrometry experiment, as is customary in proteomics. The calculation of the theoretical pI value is performed using one of the many available methods, for example the one described in Pirmoradian, M.; Zhang, B.; Chingin, K.; Astorga-Wells, J.; Zubarev, R. A. Membrane-assisted isoelectric focusing device as a micro-preparative fractionator for two dimensional shotgun proteomics, Anal. Chem, 2014, 86, 5728-5732. The process of convolution and deconvolution is summarized in FIG. 1. FIG. 1(a) is the pI-code multiplexed method according to the present disclosure while FIG. 1(b) is a standard analysis method according to prior art, in which no multiplexing is done.

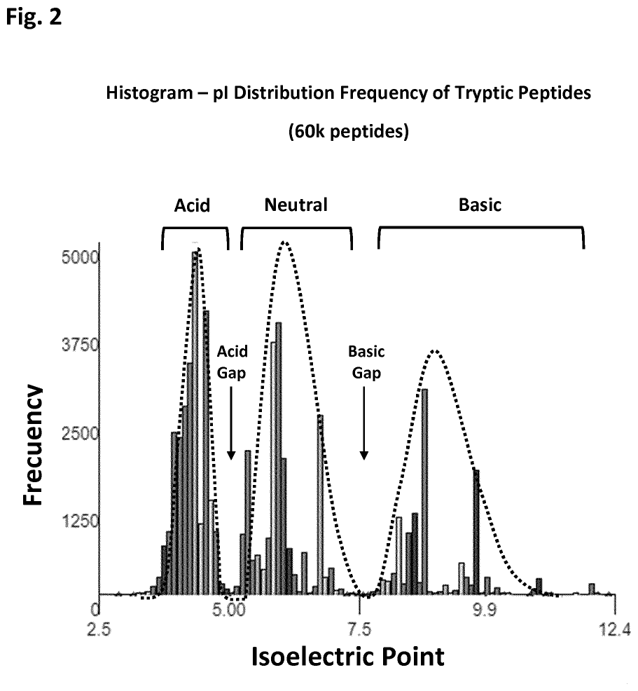

The calculations are usually accurate within a very narrow uncertainty range, such as ±0.05 pI value. Therefore, it is advantageous that the individual pI value ranges encoding the different polypeptide samples are not only non-overlapping, but are also separated by a gap of 0.05 pI units or more. As such gaps, natural empty intervals between pI values of peptides produced by a specific enzyme can be used. For instance, for unmodified tryptic peptides there are empty intervals (as depicted in FIG. 2, which is a histogram of pI distributions of tryptic peptides depicting the presence of acid, neutral and basic channel that can be used for pI-multiplexing) around the pI values 5.1+/−0.1 and 7.7+/−0.2 (isoelectric point units). In FIG. 2 the words “Acid”, “Neutral” and “Basic” (above the graph), correspond to the “Acidic Channel”, “Neutral Channel” and “Basic Channel”, respectively.

Since achieving accurate separation of polypeptides by pI may prove to be challenging (or time consuming), it is advantageous to multiplex by pI replicates of the same sample. In this manner partial pI overlap between the samples will result in a slightly enhanced measured similarity between the replicates, which is an artifact that it is more easily tolerated than pI overlap between different samples.

It is also advantageous when the number of replicates is equal to the number of encoding pI ranges. For instance, when two samples, C (control) and S (sample) are compared in two replicates 1 and 2, it is advantageous to use two pI-coding regions, A (acidic) and B (basic), obtaining fractions CA1, CA2, CB1, and CB2, for first replicate and SA1, SA2, SB1 and SB2 for the second replicate. Upon multiplexing, two pooled samples (CA1+CB2) and (SA1+SB2) are obtained. When these samples are analyzed by mass spectrometry and compared, the acidic sample polypeptides are compared with acidic control polypeptides for the first replicate, and the basic sample polypeptides are compared with basic control polypeptides for the second replicate. The advantage of the multiplexing method is that it requires two mass spectrometry analyses for obtaining two replicates instead of the conventional approach where four analyses are required, thus reducing the instrumental time by a factor of two.

In another example, when two samples C and S are compared in three replicates, it is advantageous to have three pI-coding regions, which would produce fractions CA (acidic), CN (neutral) and CB (basic) for Control and SA, SN and SB for Sample. Then the multiplexing could be done into two pooled samples (CA1+CN2+CB3) and (SA1+SN2+SB3). In the comparison between the pooled samples, acidic sample polypeptides are compared with acidic control polypeptides, neutral sample polypeptides are compared with neutral control polypeptides, and basic sample polypeptides are compared with basic control polypeptides. The advantage of the multiplexing method is that it requires two mass spectrometry analyses for obtaining three replicates instead of the conventional approach where six analyses are required, thus reducing the instrumental time by a factor of three.

The present disclosure combines multiplexed-enzymatic digestion using at least two proteolytic enzymes with isobaric labeling multiplexing reagents to improve the throughput of proteome analysis of isobaric labeled samples by mass spectrometry.

The present disclosure combines multiple non-overlapping isoelectric point ranges to encode the origin of a plurality of peptides derived from enzymatic digestions in which each isoelectric point range is populated by a sample or a set of samples.

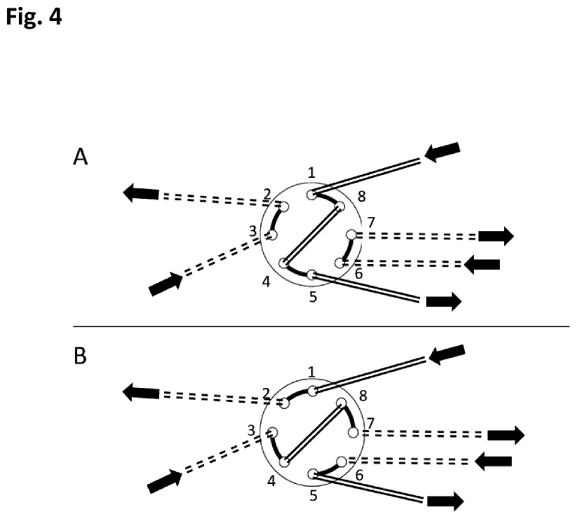

FIG. 4 illustrates an isoelectric focusing (IEF) device dedicated to isolate the Acid Channel, the Neutral Channel and the Basic Channel for pI-based multiplexing. In FIG. 4, each number corresponds to a “port number” and the arrows correspond to the direction of the flow. The isoelectric channel separation is produced by using an 8-port valve in which a first section of the device is built between the inlet and port number 1, the second section of the device is built between port number 8 and port number 4, and the third section of the IEF device is built between port number 5 and the outlet of the isoelectric focusing device. This configuration allows the possibility to collect the basic, neutral and acid fraction by running the IEF separation with the valves positioned as in FIG. 4A and collecting the fraction by switching the valve as depicted in FIG. 4B, by means of pumps connected to ports number 3 and number 6, as well as at the inlet of the tube connected to port number 1. Thus, a first fraction (either the acid channel or the basic channel, according to the electrode polarity used) may be collected from port number 2, the second fraction (the neutral channel) may be collected from port number 7, and a third fraction (either the acid channel or the basic channel, according to the electrode polarity used) may be collected from the outlet of the IEF device coming from the outlet of port number 5.

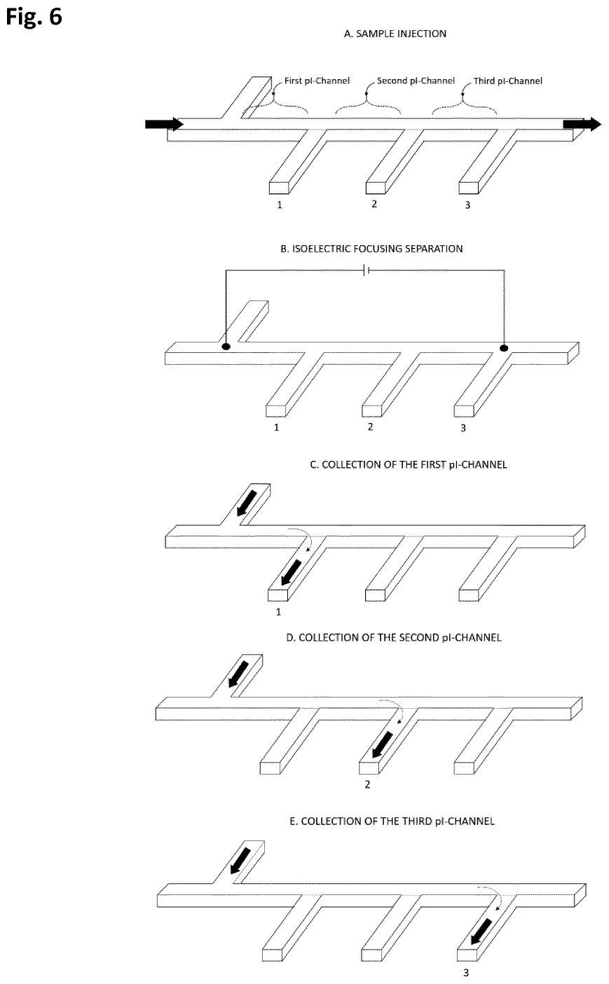

FIG. 6 illustrates schematics and operational characteristics of a device dedicated to direct isolation of pI-multiplexing channels. The device could be built as a microfluidic device or in a bigger format that matches the dimension used in standard isoelectric focusing (7 cm to 24 cm length). The operation includes sample injection (A), isoelectric focusing (IEF) separation (B) and individual collection of each pI-channel (C—first pI-channel; D—second pI-channel; and E—third pI-channel). Black arrows depict the direction of the flow. The sample containing the polypeptides is injected via hydrodynamic or air pressure into the channel in such a manner that the sample is in the First pI-Channel, Second pI-Channel and Third pI-Channel sections (FIG. 6A). After injection, an electric field provides the means for isoelectric focusing separation (FIG. 6B). After separation, the separated polypeptides are collected via the use of hydrodynamic or air pressure and valves that can be in open or closed mode, which are located on each exit of the channels. The valves are operated and pressure is applied in such a manner that the sample in the First pI-Channel, Second pI-Channel and Third pI-Channel can be collected at the exit of the each channel, respectively (FIG. 6C, 6D, 6E). The outlet of the channel for collecting the First pI-Channel is labelled with the number 1. The outlet of the channel for collecting the Second pI-Channel is labelled with the number 2. The outlet of the channel for collecting the Third pI-Channel is labelled with the number 3.

### DEFINITIONS

All words and terms used herein shall be considered to have the same meaning usually given to them by the person skilled in the art, unless another meaning is apparent from the context.
Sample. In analytical chemistry the term sample refers to a portion of material containing the analytes of interest selected from a larger quantity of material selected for analysis. Herein, the term sample is restricted to proteins or peptides from biological or synthetic origin. Non-exclusive examples are samples coming from plasma, urine, cerebrospinal fluid, saliva, tears or other biofluids. Additionally, the polypeptides could be coming from protein or peptide production systems (protein expression systems) either synthetically or those related to polypeptide systems such as solid-phase synthesis of polypeptides (e.g. t-Boc and Fmoc protecting groups). In the present disclosure the sample is processed using standard shot-gun proteomics workflow: protein extraction/solubilization (e.g. using detergent or urea, or acetone precipitation), protein reduction/alkylation, and enzymatic digestion (e.g. Lys-C, trypsin, pepsin or any other proteolytic enzyme).
Proteolytic enzyme. These enzymes catalyze the cleavage of the peptide bond in polypeptides. One important characteristic of these enzymes is specificity. Some proteins cleave polypeptide chains exclusively at the location of specific amino acids residues, such as trypsin, Lys-C and Glu-C. Others have broader specificity such as pepsin, papain and proteinase K.
Acidic channel. One of the three isoelectric point ranges in which tryptic peptides are present. This area contains polypeptides with pI values between 2.0 and 4.9 (+/−0.1). Herein, the term acidic channel can also be referred to as the first isoelectric point range. Most proteins have at least one tryptic peptide in this region.
Neutral channel: One of the three isoelectric point ranges in which tryptic peptides are present. This area contains polypeptides with pI values between 5.3 and 7.4 (+/−0.1). Herein, the term neutral channel can also be referred to as the second isoelectric point range. Most proteins have at least one tryptic peptide in this region.
Basic channel: One of the three isoelectric point ranges in which tryptic peptides are present. This area contains polypeptides with pI values between 7.7 and 12.5 (+/−0.2). Herein, the term basic channel can also be referred to as the third isoelectric point range. Most proteins have at least one tryptic peptide in this region.
Acid gap: One of the two isoelectric point ranges in which the pI frequency of tryptic peptides has a minimum value. This region has its minimum between 5.0 and 5.2 (+/−0.1) pI units. This region separates the acid channel from the neutral channel.
Neutral gap: One of the two isoelectric point ranges in which the pI frequency of tryptic peptides has a minimum value. This region has its minimum between 7.5 and 7.7 (+/−0.1) pI units. This region separates the neutral channel from the basic channel.

The general workflow according to the methods described herein may be applied to any analysis of protein samples, such as proteome samples. The at least two protein samples to be analysed by the methods described herein may be technical replicates or biological replicates. Protein samples to be analysed may undergo different treatments prior to the analysis in order to obtain different protein expression in the different samples. For example, the at least two protein samples pretreated differently may represent two different states of a proteome, and may be designated as Control and Sample. An application of the method according to the present disclosure comprises the following: Each of the Sample and Control proteome digests, with or without isobaric labeling, undergo isoelectric focusing separation, fractionating polypeptides into two distinct isoelectric focusing ranges, wherein the first range isoelectric focusing fraction of isobaric labeled or unlabeled peptides comes from the first biological or technical replicate of said proteome Sample and Control and wherein the second range isoelectric focusing fraction of isobaric labeled or unlabeled peptides comes from the first biological or technical replicate of said proteome Sample and Control, whereafter the first range of first replicate of Sample is pooled together with the second range of second replicate of Sample, while the first range of first replicate of Control is pooled together with the second range of second replicate of Control, whereafter, upon obtaining quantitative information on polypeptide abundances in each analyzed pooled sample, the abundances of polypeptides in the first isoelectric focusing range of Sample are compared to the abundances of polypeptides in the first isoelectric focusing range of Control, while the abundances of polypeptides in the second isoelectric focusing range of Sample are compared to the abundances of polypeptides in the second isoelectric focusing range of Control.

In accordance with the description and definitions above, the present disclosure is directed to the following methods:

A method for performing an analysis of a plurality of protein samples, comprising:

(a) Adding a proteolytic enzyme of a given specificity to a first protein sample to digest proteins to peptides;

(b) Separating the peptides obtained in step (a) by isoelectric focusing;

(c) Collecting those peptides which have their isoelectric point value within a first isoelectric point range;

(d) Adding a proteolytic enzyme of a given specificity to a second protein sample to digest proteins to peptides;

(e) Separating the peptides obtained in step (d) by isoelectric focusing;

(f) Collecting those peptides which have their isoelectric point value within a second isoelectric point range, where said second isoelectric point range is different and non-overlapping compared to said first isoelectric point range;

(g) Combining the peptides collected in steps (c) and (f) into a single sample and subjecting said sample to mass spectrometry analysis;

(h) Deconvoluting signals/data obtained from the mass spectrometry analysis by calculating the isoelectric point of each peptide, and assigning a peptide to the first protein sample if its isoelectric point value matches the isoelectric point range selected in step (c) or to the second protein sample if its isoelectric point value matches the isoelectric point range selected in step (f); and

(i) Obtaining quantitative information for proteins of each sample according to magnitude of the signal obtained from each peptide.

The method as described above, wherein two samples are combined, trypsin may be used as proteolytic enzyme and said first isoelectric point range is between 2 and 4.9 (+/−0.1) and said second isoelectric point range is between 5.3 and 12.8 (+/−0.1).

Alternatively, the method as described above, wherein three samples are combined by using three different non-overlapping isoelectric point ranges.

In the method as described above wherein three samples are combined, trypsin may be used as proteolytic enzyme and the first isoelectric point range which corresponds to the first sample is between 2.0 and 4.9 (+/−0.1), the second isoelectric point range which corresponds to the second sample is between 5.3 and 7.4 (+/−0.1), and the third isoelectric point range which corresponds to the third sample is between 7.7 and 12.5 (+/−0.2).

Further, in any one of the methods as described above, the following additional features may be applied:

step (a) comprises (a1) adding the proteolytic enzyme to each of a first plurality of samples to digest proteins to peptides separately in each of said first plurality of samples; (a2) adding a different isobaric label to each of said first plurality of samples to label the peptides of each sample differently; (a3) mixing said first plurality of samples to obtain a first pooled sample;

step (b) comprises isoelectric focusing of the peptides of the first pooled sample of step (a3);

step (c) comprises collecting those peptides of the first pooled sample which have their isoelectric point value within said first isoelectric point range;

step (d) comprises (d1) adding the proteolytic enzyme to each of a second plurality of samples to digest proteins to peptides separately in each of said second plurality of samples; (d2) adding a different isobaric label to each of said second plurality of samples to label the peptides of each sample differently; (d3) mixing said second plurality of samples to obtain a second pooled sample;

step (e) comprises isoelectric focusing of the peptides of the second pooled sample of step (d3);

step (f) comprises collecting those peptides of the second pooled sample which have their isoelectric point value within said second isoelectric point range;

step (g) comprises combining the peptides of the first pooled sample collected in step (c) and the peptides of the second pooled sample collected in step (f) into a single sample which is subjected to mass spectrometry; and

step (i) comprises obtaining quantitative information for proteins of each sample according to magnitude of the signal obtained from each isobaric label.

Also, the method as described above wherein a first plurality of samples and a second plurality of samples are separately digested and isobarically labelled, may further comprise the following additional features:

(f′1) adding a proteolytic enzyme to each of a third plurality of samples to digest proteins to peptides separately in each of said third plurality of samples;

(f′2) adding a different isobaric label to each of said third plurality of samples to label the peptides of each sample differently;

(f′3) mixing said third plurality of samples to obtain a third pooled sample;

(f′4) comprises isoelectric focusing of the peptides of the third pooled sample of step (f′3),

(f′5) comprises collecting those peptides of the third pooled sample which have their isoelectric point value within the third isoelectric point range; and

step (g) comprises combining the peptides of the first pooled sample collected in step (c), the peptides of the second pooled sample collected in step (f) and the peptides of the third pooled sample collected in step (f′5) into a single sample which is subjected to mass spectrometry.

Additionally, in any one of the methods as described above, the proteolytic enzyme added in step (a) and the proteolytic enzyme added in step (d) may have different and non-overlapping enzymatic specificities; in which case the deconvolution step (h) further comprises assigning a peptide to said first sample if the amino acid residue present at the N terminus or the C terminus of the peptide matches the amino acid sequence cleavage specificity of the proteolytic enzyme added to said first protein sample, or to said second protein sample if the amino acid residue present at the N terminus or the C terminus of the peptide matches the amino acid sequence cleavage specificity of the proteolytic enzyme added to said second protein sample.

The present disclosure is further directed to the following apparatuses and systems:

An apparatus for performing any one of the above-described methods, said apparatus comprising a plurality of immobilized pH gradient strips, power supplies and electrodes, characterized in that each of the immobilized pH gradient strips comprises an identification mechanism, which is able to identify a position which separates a first isoelectric point range of between 2 and 4.9 (+/−0.1) from a second isoelectric point range of between 5.3 and 12.8 (+/−0.1).

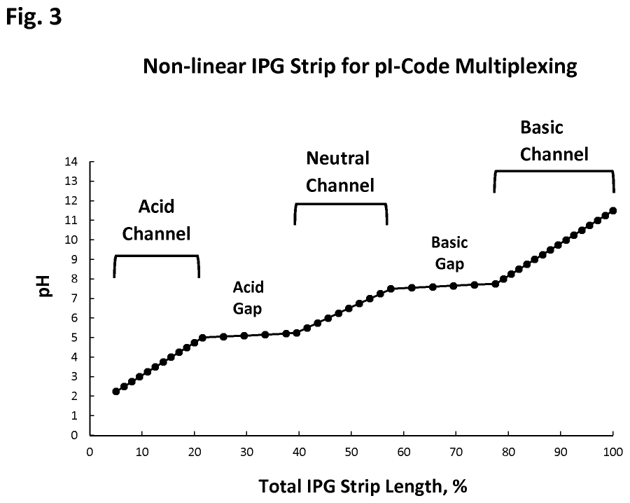

An apparatus for performing any one of the above-described methods, said apparatus comprising a plurality of non-linear immobilized pH gradient strips, power supplies and electrodes, characterized in that each of the non-linear immobilized pH gradient strips has a decreased pI variation per unit distance within an isoelectric point range between 5.0 and 5.2 (+/−0.1), and/or between 7.5 and 7.7 (+/−0.1), compared to the other isoelectric point ranges, thereby facilitating the collection of the acidic and/or neutral and/or basic isoelectric point ranges according to any one of the above-described methods. This type of apparatus may employ a non-linear immobilized pH gradient strip (IPG Strip) of the type, for which FIG. 3 shows the pH profile upon pI-Code Multiplexing.

A system for performing any one of the above-described methods, said system comprising a combination of the two apparatuses described above, i.e. comprising:

(a) an apparatus comprising a plurality of immobilized pH gradient strips, power supplies and electrodes, characterized in that each of the immobilized pH gradient strips comprises an identification mechanism, which is able to identify a position which separates a first isoelectric point range of between 2 and 4.9 (+/−0.1) from a second isoelectric point range of between 5.3 and 12.8 (+/−0.1); and

(b) an apparatus comprising a plurality of non-linear immobilized pH gradient strips, power supplies and electrodes, characterized in that each of the non-linear immobilized pH gradient strips has a decreased pI variation per unit distance within an isoelectric point range between 5.0 and 5.2 (+/−0.1), and/or between 7.5 and 7.7 (+/−0.1), compared to the other isoelectric point ranges.

An apparatus for performing any one of the above-described methods, said apparatus comprising a tube for containing a sample, a set of electrodes, ion-selective membranes to be located between the electrodes and a sample, a power supply and means to provide injection and elution of a sample to perform in-solution isoelectric focusing, and an autosampler, characterized in that the autosampler is programmed by a computer to collect peptides of the acidic isoelectric point range and/or the neutral isoelectric point range and/or the basic isoelectric point range in different vials.

An apparatus for performing any one of the above-described methods, said apparatus comprising, a plurality of fluidic channels, a set of electrodes, a plurality of ion-selective membranes located between the electrodes and the sample, wherein said plurality of fluidic channels are connected such that by closing or opening a particular set of channels and applying positive or negative pressure, the peptides of the acidic isoelectric point range and/or the peptides of the neutral isoelectric point range fraction and/or the peptides of the basic isoelectric point range fraction are mobilized into different vials.

The present disclosure will now be illustrated by the following non-limiting examples.

### EXAMPLES

### Example 1

This example uses isoelectric focusing convolution for the analysis of two samples in a single LC-MS analysis. This can be performed by the following protocol:

1. Two samples, called Sample A and Sample B, containing a mixture of proteins are separately digested with trypsin, then;

2. The resulting tryptic peptides from Sample A are separated by isoelectric focusing, then;

3. Collecting only those peptides from isoelectric point below 5. For simplicity, this sample will be called A-Acidic.

4. Perform isoelectric focusing separation of the resulting tryptic peptides from Sample B, then;

5. Collect only those peptides from isoelectric point above 5. For simplicity, this sample will be called B-Basic.

6. Mix together samples A-Acidic and B-Basic. For simplicity this sample will be called ABplexed.

7. Perform LC MS/MS analysis of ABplexed.

8. Perform database search of the LC MS/MS data (protein and peptide identification and quantification), and calculating the isoelectric point of each identified peptide.

9. Perform data deconvolution by assigning to Sample A only those peptides with isoelectric point lower than 5, and assigning to Sample B only those peptides with isoelectric point higher and 5.

10. Perform protein quantification of Sample A and Sample B by standard label free quantification.

### Example 2

This example uses isoelectric focusing convolution for the analysis of two isobaric labeled samples in a single LC-MS analysis (in this example, in total 16 samples).

1. Eight samples, each containing a mixture of proteins, are individually and separately digested with trypsin. After digestion, each digest is later labeled with a different isobaric reagent in a manner that when mixed together (or pooled), it will allow the quantification of each individual protein sample. Each labeled sample will be called Sample A1, Sample A2, Sample A3, Sample A4, Sample A5, Sample A6 Sample A7 and Sample A8, respectively.

2. Mix together (pool) samples: Sample A1, Sample A2, Sample A3, Sample A4, Sample A5, Sample A6, Sample A7 and Sample A8 into a single sample. For simplicity, this pooled sample will be called A-8plexed

3. Perform isoelectric focusing separation to the A-8plexed sample.

4. Collect only those peptides from isoelectric point below 5. For simplicity, this sample will be called A-8plexed-Acidic.

5. Another eight samples, each containing a mixture of proteins are individually and separately digested with trypsin, and the resulting peptides are each labeled with a different isobaric reagent in a manner that when mixed together (or pooled), it will allow the quantification of each individual protein sample. The isobaric reagent might be the same as the one used in the previous steps (steps 1 to 3 on Examples claim 2). Each labeled sample will be called Sample B1, Sample B2, Sample B3, Sample B4, Sample B5, Sample B6 Sample B7 and Sample B8, respectively.

6. Mix together (pool) samples: Sample B1, Sample B2, Sample B3, Sample B4, Sample B5, Sample B6 Sample B7 and Sample B8 into a single sample. For simplicity, this pooled sample will be called B-8plexed.

7. Perform isoelectric focusing separation to the B-8plexed sample.

8. Collect only those peptides from isoelectric point above 5. For simplicity, this sample will be called B-8plexed-Basic.

9. Mixing together the samples A-8plexed-Acidic and B-8plexed-Basic. For simplicity this sample will be called AB-8plexed.

10. Perform a LC MS/MS analysis of the AB-8plexed sample.

11. Perform database search of the LC MS/MS data (protein and peptide identification and quantification), and calculating the isoelectric point of each identified peptide.

12. Perform data deconvolution by assigning to A-8plexed-Acidic only those peptides with isoelectric point lower than 5, and assigning to B-8plexed-Basic only those peptides with isoelectric point higher and 5.

13. Perform protein quantification by standard isobaric labelling quantification for the quantification of Sample A1, Sample A2, Sample A3, Sample A4, Sample A5, Sample A6 Sample A7 and Sample A8 and Sample B1, Sample B2, Sample B3, Sample B4, Sample B5, Sample B6 Sample B7 and Sample B8.

### Example 3

Combination of isoelectric focusing convolution and multi-enzyme convolution, allowing the analysis of 4 label free samples in a single LC-MS run.

1. One sample, called Sample A containing a mixture of proteins is digested with trypsin, then;

2. Another sample, called Sample B containing a mixture of proteins is digested with pepsin or any other proteolytic enzyme having a different and orthogonal enzymatic specificity than trypsin, then;

3. Another sample, called Sample C containing a mixture of proteins is digested with trypsin, then;

4. Another sample, called Sample D containing a mixture of proteins is digested with pepsin or any other proteolytic enzyme having a different and orthogonal enzymatic specificity than trypsin, then;

5. Mix the resulting peptides from Sample A and Sample B in a single sample. For simplicity, this sample will be called Sample AtBp.

6. Perform isoelectric focusing separation to the AtBp sample, and collect only those peptides from isoelectric point below 5. For simplicity, this sample will be called AtBp-Acidic.

7. Mix the resulting peptides from Sample C and Sample D in a single sample. For simplicity, this sample will be called Sample CtDp.

8. Perform isoelectric focusing separation to the CtDp sample, and collect only those peptides from isoelectric point above 5. For simplicity, this sample will be called CtDp-Basic.

9. Mix together samples AtBp-Acidic and CtDp-Basic. For simplicity this sample will be called ABCDplexed.

10. Perform LC MS/MS analysis of ABCDplexed.

11. Perform database search of the LC MS/MS data (protein and peptide identification and quantification), and calculating the isoelectric point of each identified peptide.

12. Perform data deconvolution by assigning to Sample A and Sample B only those peptides with isoelectric point lower than 5, and assigning to Sample C and Sample D only those peptides with isoelectric point higher and 5.

13. Perform data deconvolution by assigning to Sample A only those peptides coming from trypsin digestion (an arginine or a lysine residue at the C terminal of the peptide) and with isoelectric point lower than 5.

14. Perform data deconvolution by assigning to Sample C only those peptides coming from trypsin digestion (an arginine or a lysine residue at the C terminal of the peptide) and with isoelectric point higher than 5.

15. Perform data deconvolution by assigning to Sample B only those peptides not coming from trypsin digestion (peptides must not have an arginine or a lysine residue at the C terminal of the peptide, and preferable phenylalanine, leucine, methionine, cysteine, glutamate, aspartate and tryptophan, alanine and glutamine) and with isoelectric point lower than 5.

16. Perform data deconvolution by assigning to Sample D only those peptides not coming from trypsin digestion (peptides must not have an arginine or a lysine residue at the C terminal of the peptide, and preferable phenylalanine, leucine, methionine, cysteine, glutamate, aspartate and tryptophan, alanine and glutamine) and with isoelectric point higher than 5.

17. Perform protein label free quantification to Sample A, Sample B, Sample C and Sample D.

### Example 4

Combination of isoelectric focusing convolution, multi-enzyme convolution and isobaric labelling, allowing the analysis of 4 isobaric labeled samples in a single LC-MS analysis (in this example, in total 32 samples).

1. Eight samples, each containing a mixture of proteins, are individually and separately digested with trypsin, and the resulting peptides are each labeled with a different isobaric reagent in a manner that when mixed together (or pooled), it will allow the quantification of each individual protein sample. For simplicity, each labeled sample will be called Sample At1, Sample At2, Sample At3, Sample At4, Sample At5, Sample At6 Sample At7 and Sample At8, respectively (in this particular sample nomenclature, the “t” refers to trypsin).

2. Mix together (pool) samples: Sample At1, Sample At2, Sample At3, Sample At4, Sample At5, Sample At6 Sample At7 and Sample At8 into a single sample. For simplicity, this pooled sample will be called At-8plexed.

3. Another eight samples, each containing one or more proteins, are individually and separately digested with pepsin or any other proteolytic enzyme having a different and orthogonal enzymatic specificity than trypsin; and the resulting peptides are each labeled with a different isobaric reagent in a manner that when mixed together (or pooled), it will allow the quantification of each individual protein sample. The isobaric reagent might be the same as the one used in the previous step (steps 1 in Examples claim 4). For simplicity, each labeled sample will be called Sample Bp1, Sample Bp2, Sample Bp3, Sample Bp4, Sample Bp5, Sample Bp6 Sample Bp7 and Sample Bp8, respectively (in this particular sample nomenclature, the “p” refers to pepsin).

4. Mix together (pool) samples: Sample Bp1, Sample Bp2, Sample Bp3, Sample Bp4, Sample Bp5, Sample Bp6 Sample Bp7 and Sample Bp8 into a single sample. For simplicity, this pooled sample will be called Bp-8plexed.

5. Another eight samples, each containing a mixture of proteins are individually and separately digested with trypsin, and the resulting peptides are each labeled with a different isobaric reagent in a manner that when mixed together (or pooled), it will allow the quantification of each individual protein sample. For simplicity, each labeled sample will be called Sample Ct1, Sample Ct2, Sample Ct3, Sample Ct4, Sample Ct5, Sample Ct6 Sample Ct7 and Sample Ct8, respectively (in this particular sample nomenclature, the “t” refers to trypsin).

6. Mix together (pool) samples: Sample Ct1, Sample Ct2, Sample Ct3, Sample Ct4, Sample Ct5, Sample Ct6 Sample Ct7 and Sample Ct8 into a single sample. For simplicity, this pooled sample will be called Ct-8plexed.

7. Another eight samples, each containing one or more proteins are individually and separately digested with pepsin or any other proteolytic enzyme having a different and orthogonal enzymatic specificity than trypsin; and the resulting peptides are each labeled with a different isobaric reagent in a manner that when mixed together (or pooled), it will allow the quantification of each individual protein sample. The isobaric reagent might be the same as the one used in the previous step (steps 1 in Examples claim 4). For simplicity, each labeled sample will be called Sample Dp1, Sample Dp2, Sample Dp3, Sample Dp4, Sample Dp5, Sample Dp6 Sample Dp7 and Sample Dp8, respectively (in this particular sample nomenclature, the “p” refers to pepsin).

8. Mix together (pool) samples: Sample Dp1, Sample Dp2, Sample Dp3, Sample Dp4, Sample Dp5, Sample Dp6 Sample Dp7 and Sample Dp8 into a single sample. For simplicity, this pooled sample will be called Dp-8plexed.

9. Mix together (pool) samples: At-8plexed with Bp-8plexed. For simplicity, this pooled sample will be called AtBp-8plexed.

10. Perform isoelectric focusing separation to the AtBp-8plexed sample, and collect only those peptides from isoelectric point below 5. For simplicity, this sample will be called AtBp-8plexed-Acidic.

11. Mix together (pool) samples: Ct-8plexed with Dp-8plexed. For simplicity, this pooled sample will be called CtDp-8plexed.

12. Perform isoelectric focusing separation to the CtDp-8plexed sample, and collect only those peptides from isoelectric point above 5. For simplicity, this sample will be called CtDp-8plexed-Basic.

13. Mix together (pool) samples: AtBp-8plexed-Acidic with CtDp-8plexed-Basic. For simplicity, this pooled sample will be called AtBpCtDp-8plexed.

14. Perform LC MS/MS analysis of AtBpCtDp-8plexed sample.

15. Perform database search of the LC MS/MS data (protein and peptide identification and quantification), and calculating the isoelectric point of each identified peptide.

16. Perform data deconvolution by assigning to Sample A only those peptides coming from trypsin digestion (an arginine or a lysine residue at the C terminal of the peptide) and with isoelectric point lower than 5.

17. Perform data deconvolution by assigning to Sample B only those peptides not coming from trypsin digestion (peptides must not have an arginine or a lysine residue at the C terminal of the peptide, and preferable phenylalanine, leucine, methionine, cysteine, glutamate, aspartate and tryptophan, alanine and glutamine) and with isoelectric point lower than 5.

18. Perform data deconvolution by assigning to Sample C only those peptides coming from trypsin digestion (an arginine or a lysine residue at the C terminal of the peptide) and with isoelectric point higher than 5.

19. Perform data deconvolution by assigning to Sample D only those peptides not coming from trypsin digestion (peptides must not have an arginine or a lysine residue at the C terminal of the peptide, but preferably contain preferable phenylalanine, leucine, methionine, cysteine, glutamate, aspartate and tryptophan, alanine and glutamine) and with isoelectric point lower than 5.

20. Perform protein quantification by standard isobaric labelling quantification of the peptides assigned in the previous steps for the quantification of Sample A1, Sample A2, Sample A3, Sample A4, Sample A5, Sample A6, Sample A7 and Sample A8, as well as Sample B1, Sample B2, Sample B3, Sample B4, Sample B5, Sample B6, Sample B7, Sample B8, as well as Sample Ct1, Sample Ct2, Sample Ct3, Sample Ct4, Sample Ct5, Sample Ct6, Sample Ct7 and Sample Ct8, as well as Sample Dp1, Sample Dp2, Sample Dp3, Sample Dp4, Sample Dp5, Sample Dp6, Sample Dp7 and Sample Dp8.

### Example 5

### Materials and Methods

A three-channel isoelectric point-based multiplexing was applied for the analysis of proteome changes in protein abundance upon drug treatment, using the following procedure:

Human Colon Carcinoma Cells HCT116 were cultured at 37° C. with 5% CO2 in high-glucose Dulbecco's Modified Eagle's Medium (DMEM, Thermo Fisher Scientific) supplemented with 10% fetal bovine serum (Gibco) and 1% penicillin/streptomycin (Gibco). The cells were treated for 48 h with 45 nM Methotrexate in 0.01% dimethyl sulfoxide (DMSO). As a control experiment, cells were treated with 0.01% DMSO. The medium containing the drug (or 0.01% DMSO for the control) was replaced each 24 h by fresh medium. A total of 3 control samples and 3 treated samples were produced.

Cells (1 million cells per replicate sample) were washed twice with PBS (1 mL) and resuspended in 300 μL of lysis buffer (3% SDC, 20 mM EPPS, pH 8.5). The total protein concentration was measured using the BCA protein assay kit (Pierce) in accordance with the manufacturer's protocol. The extracted proteins were reduced with 15 mM dithiothreitol (DTT) for 30 min at 60° Celsius and subsequently alkylated with 20 mM iodoacetamide (IAA) for 45 min in the dark. The concentration of SDC was decreased to 1.5% with 20 mM EPPS buffer pH 8.5 and digested with 2 μg modified sequencing grade trypsin (Promega). After 14 h of tryptic digestion, the reaction was stopped with acetic acid to a final concentration of 5% w/w incubated for 30 min followed by a 15 min/20000 g centrifugation. Samples were cleaned using C-18 SepPack (Waters), and the eluted peptides were dried in a SpeedVac centrifugal evaporator.

Isoelectric Focusing. In-solution isoelectric focusing separation was performed using a pI-Trap instrument (Biomotif AB). The instrument performs in-solution IEF, and its operation and configuration has been described elsewhere (Pirmoradian M et al 2015, Pirmoradian M et al 2014, Chingin K at al 2012). The following protocol was performed for every single sample (control and treated):

1. Tryptic peptides were dissolved in 2% ampholyte (pI range 3-10, GE HealthCare), and separated using a 210 μA current-limited method for 1 hr (voltage varied between 0.9 to 1400 kV). Fractions were collected every 1 min at 0.5 μL/min for 25 min.

2. For each individual replicate of the Control and Treated samples, 3 fractions were generated according to the following isoelectric point values of the tryptic peptides in each fraction collected after isoelectric focusing.

Acidic Channel: pI between 2.0 and 5.0),

Neutral Channel: pI between 5.3 and 7.4), and

Basic Channel: pI between 7.7 and 12.5).

3. A single pI-multiplexed control sample was generated by combining the Acidic Channel from the first biological replicate, the Neutral Channel from the second biological replicate and the Basic Channel from the third biological replicate. Thus, this individual pI-multiplexed sample contains 3 biological replicates.

4. A single pI-multiplexed treated sample was generated by combining the Acidic Channel from the first biological replicate, the Neutral Channel from the second biological replicate and the Basic Channel from the third biological replicate. Thus, this individual pI-multiplexed sample contains 3 biological replicates.

5. A LC MS/MS analysis was performed to each multiplexed sample under a 240 min gradient using a 50 cm EasySpray C-18 column at 300 nL/min using a 0.1% formic acid and acetonitrile gradient (5% to 95% in 240 min).

6. Samples were analyzed together within MaxQuant (1% FDR and match between runs) and Quanty quantification software (1% FDR). Isoelectric point calculations were in-silico calculated as described in Pirmoradian M et al 2014. Proteins containing at least 1 peptide on each pI-channel were selected for quantification.

### RESULTS

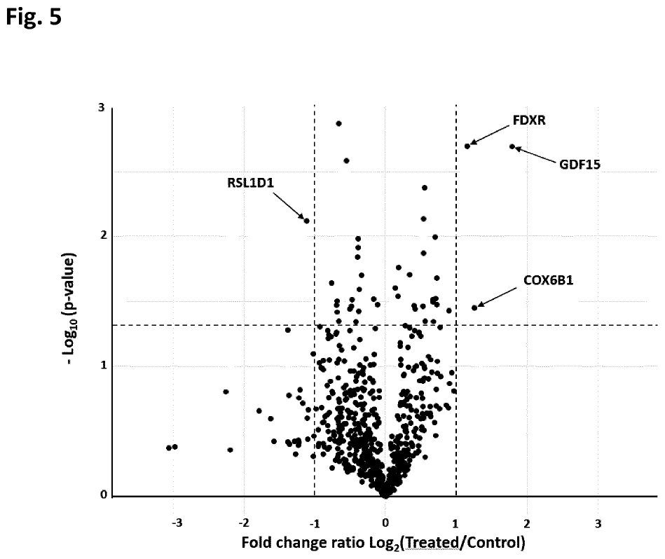

After pI-coding signal deconvolution, 570 proteins were quantified over 6 deconvolutes samples, involving 3 Control and 3 Treated samples. Since each deconvoluted Control and Treated sample contains 3 biological replicates it is possible to obtain protein quantification data suitable for statistical analysis. To graphically represent statistically significant data, a volcano plot—log₁₀(P value) vs. log₂(fold change of Treated/Control)—was constructed to graphically display changes in protein abundance between control and treated sample by use of 3-channel pI-multiplexing (FIG. 5). Points above the dashed horizontal line represent proteins with significantly different abundances (p<0.05) between control and treated sample. Points to the left of the left-most dashed vertical line denote protein fold changes of Treated/Control less than −1.0, while points to the right of the right-most dashed vertical line denote protein fold changes of Treated/Control greater than 1.0. In total, 4 proteins demonstrated a statistical difference between the control and treated. From these proteins, FDXR, GDF15, and COX6B1 have been reported to be sensitive to changes upon anticancer drugs Methotrexate and 5-FU (Chernobrovkin A, et al 2015 and Marin-Vicente C et al.).

### REFERENCES

Pirmoradian M, Astorga-Wells J, Zubarev R A. Anal Chem. 2015 Dec. 1;87(23):11840-6. doi: 10.1021/acs.analchem.5b03344. Epub 2015 Nov. 12.
Pirmoradian M, Zhang B, Chingin K, Astorga-Wells J, Zubarev R A. Anal Chem. 2014 Jun. 17;86(12):5728-32. doi: 10.1021/ac404180e. Epub 2014 May 28.
Chingin K, Astorga-Wells J, Pirmoradian Najafabadi M, Lavold T, Zubarev R A. Anal Chem. 2012 Aug. 7;84(15):6856-62. doi: 10.1021/ac3013016. Epub 2012 Jul. 23.
Chernobrovkin A, Marin-Vicente C, Visa N, Zubarev R A. Sci Rep. 2015 Jun. 8;5:11176. doi: 10.1038/srep11176.
Marin-Vicente C, Lyutvinskiy Y, Romans Fuertes P, Zubarev R A, Visa N. J Proteome Res. 2013 Apr. 5;12(4):1969-79. doi: 10.1021/pr400052p. Epub 2013 Mar. 21.

1. A method for performing an analysis of a plurality of protein samples, comprising:
(a) adding a proteolytic enzyme of a given specificity to a first protein sample to digest proteins to peptides;
(b) separating the peptides obtained in step (a) by isoelectric focusing;
(c) collecting those peptides which have their isoelectric point value within a first isoelectric point range;
(d) adding a proteolytic enzyme of a given specificity to a second protein sample to digest proteins to peptides;
(e) separating the peptides obtained in step (d) by isoelectric focusing;
(f) collecting those peptides which have their isoelectric point value within a second isoelectric point range, where said second isoelectric point range is different and non-overlapping compared to said first isoelectric point range;
(g) combining the peptides collected in steps (c) and (f) into a single sample and subjecting said sample to mass spectrometry analysis;
(h) deconvoluting signals/data obtained from the mass spectrometry analysis by calculating the isoelectric point of each peptide, and assigning a peptide to the first protein sample if its isoelectric point value matches the isoelectric point range selected in step (c) or to the second protein sample if its isoelectric point value matches the isoelectric point range selected in step (f); and
(i) obtaining quantitative information for proteins of each sample according to magnitude of the signal obtained from each peptide.

2. The method according to claim 1, wherein trypsin is used as proteolytic enzyme and said first isoelectric point range is between 2 and 4.9 (+/−0.1) and said second isoelectric point range is between 5.3 and 12.8 (+/−0.1).

3. The method according to claim 1, wherein three samples are combined by using three different non-overlapping isoelectric point ranges.

4. The method according to claim 3, wherein trypsin is used as proteolytic enzyme and the first isoelectric point range which corresponds to the first sample is between 2.0 and 4.9 (+/−0.1), the second isoelectric point range which corresponds to the second sample is between 5.3 and 7.4 (+/−0.1), and the third isoelectric point range which corresponds to the third sample is between 7.7 and 12.5 (+/−0.2).

5. The method according to claim 1, wherein:
step (a) comprises (a1) adding the proteolytic enzyme to each of a first plurality of samples to digest proteins to peptides separately in each of said first plurality of samples; (a2) adding a different isobaric label to each of said first plurality of samples to label the peptides of each sample differently;
(a3) mixing said first plurality of samples to obtain a first pooled sample;
step (b) comprises isoelectric focusing of the peptides of the first pooled sample of step (a3);
step (c) comprises collecting those peptides of the first pooled sample which have their isoelectric point value within said first isoelectric point range;
step (d) comprises (d1) adding the proteolytic enzyme to each of a second plurality of samples to digest proteins to peptides separately in each of said second plurality of samples; (d2) adding a different isobaric label to each of said second plurality of samples to label the peptides of each sample differently; (d3) mixing said second plurality of samples to obtain a second pooled sample;
step (e) comprises isoelectric focusing of the peptides of the second pooled sample of step (d3);
step (f) comprises collecting those peptides of the second pooled sample which have their isoelectric point value within said second isoelectric point range;
step (g) comprises combining the peptides of the first pooled sample collected in step (c) and the peptides of the second pooled sample collected in step (f) into a single sample which is subjected to mass spectrometry; and
step (i) comprises obtaining quantitative information for proteins of each sample according to magnitude of the signal obtained from each isobaric label.

6. The method according to claim 5, further comprising:
(f′1) adding a proteolytic enzyme to each of a third plurality of samples to digest proteins to peptides separately in each of said third plurality of samples;
(f′2) adding a different isobaric label to each of said third plurality of samples to label the peptides of each sample differently;
(f′3) mixing said third plurality of samples to obtain a third pooled sample;
(f′4) comprises isoelectric focusing of the peptides of the third pooled sample of step (f′3);
(f′5) comprises collecting those peptides of the third pooled sample which have their isoelectric point value within the third isoelectric point range; and
step (g) comprises combining the peptides of the first pooled sample collected in step (c), the peptides of the second pooled sample collected in step (f) and the peptides of the third pooled sample collected in step (f′5) into a single sample which is subjected to mass spectrometry.

7. The method according to claim 1, wherein
the proteolytic enzyme added in step (a) and the proteolytic enzyme added in step (d) have different and non-overlapping enzymatic specificities; and the deconvolution step (h) further comprises assigning a peptide to said first sample if the amino acid residue present at the N terminus or the C terminus of the peptide matches the amino acid sequence cleavage specificity of the proteolytic enzyme added to said first protein sample, or to said second protein sample if the amino acid residue present at the N terminus or the C terminus of the peptide matches the amino acid sequence cleavage specificity of the proteolytic enzyme added to said second protein sample.

8. An apparatus for performing the method of claim 1, said apparatus comprising a plurality of immobilized pH gradient strips, power supplies and electrodes, characterized in that each of the immobilized pH gradient strips comprises an identification mechanism, which is able to identify a position which separates a first isoelectric point range of between 2 and 4.9 (+/−0.1) from a second isoelectric point range of between 5.3 and 12.8 (+/−0.1).

9. An apparatus for performing the method of claim 1, said apparatus comprising a plurality of non-linear immobilized pH gradient strips, power supplies and electrodes, characterized in that each of the non-linear immobilized pH gradient strips has a decreased pI variation per unit distance within an isoelectric point range between 5.0 and 5.2 (+/−0.1), and/or between 7.5 and 7.7 (+/−0.1), compared to the other isoelectric point ranges, thereby facilitating the collection of the acidic and/or neutral and/or basic isoelectric point ranges according to the method of any one of claims 1 to 7.

10. An apparatus for performing the method of claim 1, said apparatus comprising a tube for containing a sample, a set of electrodes, ion-selective membranes to be located between the electrodes and a sample, a power supply and means to provide injection and elution of a sample to perform in-solution isoelectric focusing, and an autosampler, characterized in that the autosampler is programmed by a computer to collect peptides of the acidic isoelectric point range and/or the neutral isoelectric point range and/or the basic isoelectric point range in different vials.
